(19) United States
(12) Patent Application Publication
(10) Pub. No.: US 2024/0350099 A1
(43) Pub. Date: Oct. 24, 2024

(54) DETECTION OF INCONTINENCE EVENTS

(71) Applicant: Hill-Rom Services, Inc., Batesville, IN (US)

(72) Inventors: John A. Lane, Venice, FL (US); WonKyung McSweeney, Manlius, NY (US); David E. Quinn, Sennett, NY (US); Harsh Dweep, Buffalo Grove, IL (US); Christopher Nelson, Longmont, CO (US)

(21) Appl. No.: 18/637,597

(22) Filed: Apr. 17, 2024

Related U.S. Application Data
(60) Provisional application No. 63/497,668, filed on Apr. 21, 2023.

(51) Int. Cl. A61B 5/00; A61B 5/01; A61G 7/10; G06T 7/00

(52) U.S. Cl. CPC A61B 5/7282; A61B 5/0077; A61B 5/01; A61B 5/742; A61G 7/1001; G06T 7/0012; G06T 2207/10016; G06T 2207/20084

(57) ABSTRACT

A patient monitoring system includes a computing device that having a processor and a memory, the memory comprising instructions that, when executed, cause the system to receive, from a thermographic camera, at least one image corresponding to a temperature of a region of interest and a surrounding area that surrounds the region of interest; use an artificial intelligence model to analyze the at least one image; detect a difference in temperature of the region of interest relative to the surrounding area; and issue an alert related to an incontinence event when the difference in temperature exceeds a threshold value.

### BACKGROUND

Patients with incontinence issues experience a reduced ability, or inability, to control bladder functions or bowel movements. This may lead to involuntary wetting or soiling of the patient's coverings. Patients are at risk for developing injuries or infection if their coverings remain wet or soiled for an extended period. Caregivers often check patients' coverings for wetting or soiling in addition to performing other tasks. Caregivers may check the patient's coverings by communicating with the patient or by removing the patient's coverings to manually check for wetting or soiling.

The number of times a patient's coverings are checked for wetting or soiling is impacted by the availability of caregivers, who are often limited by the amount of time they can spend helping any individual patient. Patients that experience incontinence may require consistent attention, which increases patient cost and the labor required from caregivers.

### SUMMARY

In general terms, the present disclosure relates to the detection of incontinence events. In one possible configuration and by non-limiting example, this can include detection of risk factors for patient deterioration within an observed area without physically contacting a patient. Various aspects are described in this disclosure, which include, but are not limited to, the following aspects.

In one aspect, a patient monitoring system includes a computing device that having a processor and a memory, the memory comprising instructions that, when executed, cause the system to receive, from a thermographic camera, at least one image corresponding to a temperature of a region of interest and a surrounding area that surrounds the region of interest; use an artificial intelligence model to analyze the at least one image; detect a difference in temperature of the region of interest relative to the surrounding area; and issue an alert related to an incontinence event when the difference in temperature exceeds a threshold value.

In another aspect, a method for detecting and mitigating patient deterioration includes receiving, from a thermographic camera, at least one image corresponding to a temperature of a region of interest and a surrounding area that surrounds the region of interest; using an artificial intelligence model, analyzing the at least one image; detecting a difference in temperature of the region of interest relative to the surrounding area; and issuing an alert related to an incontinence event when the difference in temperature exceeds a threshold value

### DESCRIPTION OF THE FIGURES

The following drawing figures, which form a part of this application, are illustrative of the described technology and are not meant to limit the scope of the disclosure in any manner.

FIG. 1 schematically illustrates an example patient monitoring system.

FIG. 2 is a block diagram of an example monitoring device of the system of FIG. 1, including a video module and a motion module.

FIG. 3 is a block diagram of the example video module of the monitoring device of FIG. 2.

FIG. 4 is a block diagram of the example motion module of the monitoring device of FIG. 2.

FIG. 5 is a block diagram of an example computing device of the system of FIG. 1.

FIG. 6 illustrates an example image captured by the monitoring device of FIG. 2 showing an incontinence event.

FIG. 7 illustrates another example incontinence event of a subject S that is detected by the monitoring device of FIG. 2 below a patient covering.

FIG. 8 illustrates a method for detecting and mitigating patient deterioration.

FIG. 9 illustrates a method of associating a region of interest with one or more patient risk factors.

FIG. 10 illustrates an exemplary architecture of the computing device of FIG. 5.

### DETAILED DESCRIPTION

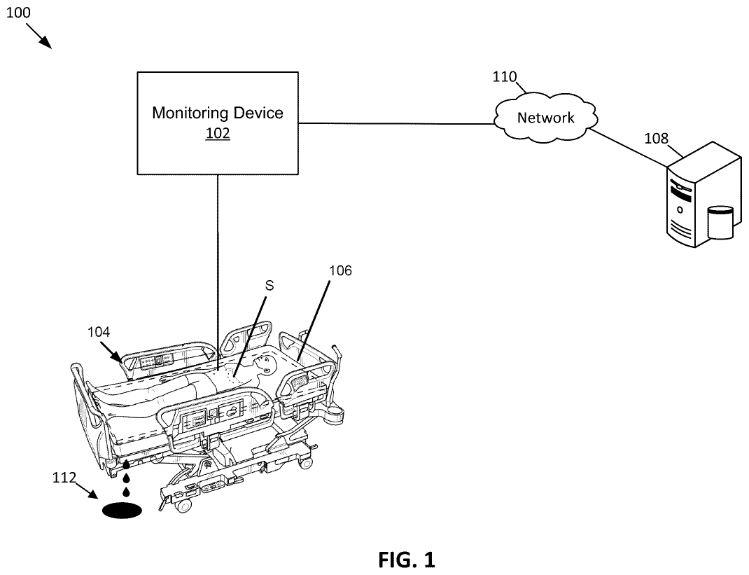

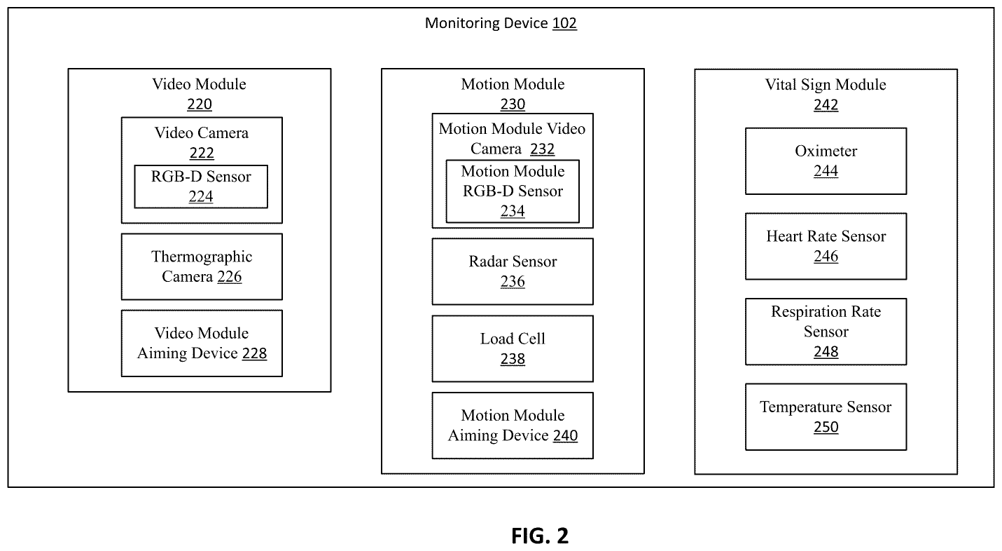

FIG. 1 schematically illustrates an example patient monitoring system 100. As shown, the patient monitoring system 100 includes a monitoring device 102 configured to observe the patient. In some embodiments, the monitoring device records a chronological series of frames including a plurality of images using a video module 220 or a motion module 230. The monitoring device 102 is illustrated and described in further detail with respect to FIGS. 2-4.

The subject S is a person, such as a patient, who is clinically treated by one or more caregivers. As shown in FIG. 1, the subject S is arranged in the subject arrangement area 104, which can be located inside a healthcare facility such as a hospital, medical clinic, doctor's office, etc. The subject arrangement area can include objects used to treat or provide comfort to the subject S, including a bedsheet, clothes, blankets, or the like. The subject arrangement area 104 can include a support device 106, such as a bed, on which the subject S can lie, rest, or sleep. Other examples of the support device 106 include lifts, chairs, stretchers, and surgical tables.

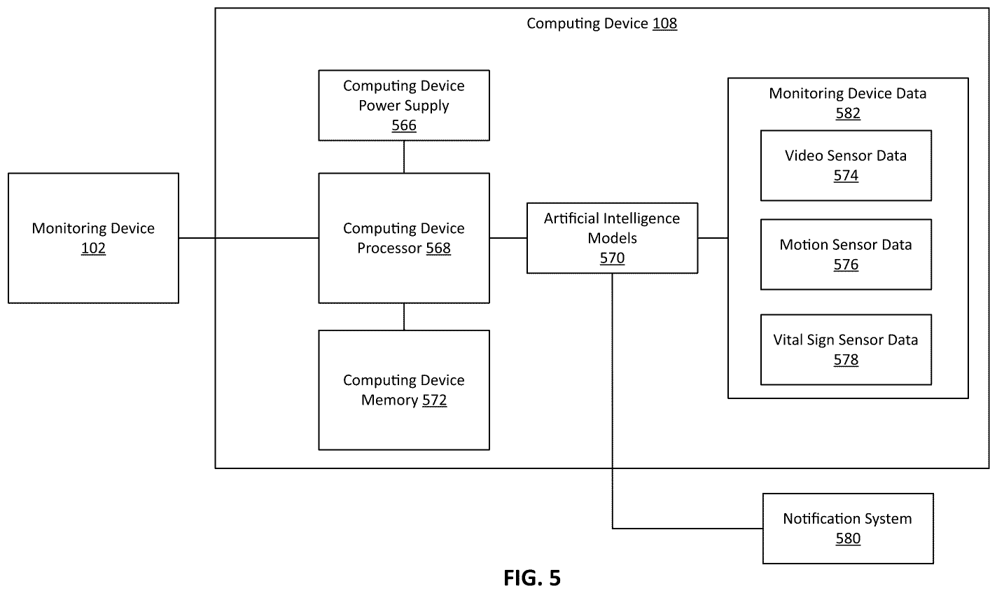

In some examples, the monitoring device 102 is operable to communicate with an example computing device 108 via a data communication network 110. The computing device 108 includes a processor 114, a memory 116, and a power supply 118. The computing device 108 includes a processing device and at least one non-transitory computer readable data storage device storing instructions executable by the processing device. The memory includes instructions that, when executed, cause the patient monitoring system 100 to perform a plurality of functions as described herein in further detail with respect to FIGS. 2-9. The computing device 108 is illustrated and described in further detail with respect to FIG. 5.

The data communication network 110 communicates data between one or more computing devices, such as among the monitoring device 102 and the computing device 108. Examples of the network 110 include a local area network and a wide area network, such as the Internet. In some examples, the network 110 includes a wireless communication system, a wired communication system, or a combination of wireless and wired communication systems. A wired communication system can transmit data using electrical or optical signals in various possible configurations. Wireless communication systems typically transmit signals via electromagnetic waves, such as in the form of optical signals or radio frequency (RF) signals.

A wireless communication system can include an optical or RF transmitter for transmitting optical or RF signals, and an optical or RF receiver for receiving optical or RF signals. Examples of wireless communication systems include Wi-Fi communication devices that utilize wireless routers or wireless access points, cellular communication devices that utilize one or more cellular base stations, Bluetooth, ANT, ZigBee, medical body area networks, personal communications service (PCS), wireless medical telemetry service (WMTS), and other wireless communication devices and services.

FIG. 2 is a block diagram of the example monitoring device 102 of FIG. 1, including one or more of a video module 220, a motion module 230, and a vital sign module 242. Each of the video module 220, the motion module 230, and the vital sign module 242 may function alone or in conjunction with other components of the monitoring device 102, as described herein.

The video module 220 is configured to capture a video including a plurality of images in a chronological sequence and transmit the video to the computing device 108. In some embodiments, the video module 220 includes a video camera 222. The video camera 222 is configured to capture video using an additive RGB (red, blue, and green) color model. The video camera 222 includes an RGB-D sensor to further provide per-pixel depth information to an RGB image. In some embodiments, the video module 220 includes a thermographic camera 226 configured to capture thermal images that convert infrared radiation into visible images to depict a spatial distribution of temperature. The thermographic camera 226 detects thermal radiation of any object within its view to generate a heat landscape that can be analyzed by the computing device 108.

The video module 220 also includes a video module aiming device 228 configured to adjust the view of the video module 220 by aiming the video module 220 at a region of interest and a surrounding area. In some embodiments, the video module aiming device 228 includes a manual adjustment for adjusting the view of the video module 220. In some embodiments, the video module aiming device 228 includes an automatic adjustment for automatically adjusting the view of the video module 220. The region of interest is an area that could potentially include a patient deterioration event such as an incontinence event. The surrounding area is an area surrounding the region of interest.

The video module aiming device 228 is configured to aim the video module 220 at the region of interest by receiving instructions from the computing device 108. Upon receiving the instructions from the computing device 108, the thermographic camera 226 is configured to measure the current temperature of the region of interest relative to the surrounding area in a plurality of images that include thermographic data. The thermographic camera 226 then transmits the plurality of images to the computing device 108 for further analysis.

In some embodiments, the instructions direct the video module aiming device 228 to a predetermined location that includes the region of interest. The predetermined location can include the subject S, the subject arrangement area 104, the support device 106, or a safety hazard 112. Safety hazard 112 include any event that present a risk to the subject's safety or general patient wellbeing within a patient care facility. Safety hazards can include spill incidents that create a fall risk from solid and/or liquid materials that are left on the floor in a patient care facility. Non-limiting examples of spill incidents may occur from vomiting, spitting, bleeding, or dropping food or beverages.

In a non-limiting example, the region of interest may include a predetermined location that includes the subject S. Specifically, the predetermined location can include the groin area of the subject S to detect an incontinence event. The thermographic camera 226 can detect changes in temperature within the predetermined location. The computing device 108 can receive information from the thermographic camera to analyze the changes in temperature and form a prediction that an incontinence event has occurred based on the increase in temperature within the predetermined location (i.e., the groin area of the subject S). If a sudden increase in temperature is viewed within the predetermined location, then the computing module 108 can predict with greater confidence that it is due to an incontinence event.

In some embodiments, the instructions direct the video module aiming device 228 to a view where the thermographic camera 226 observes an increase in temperature (i.e., the computing device 108 can instruct the video module 220 to change its view to a new region of interest if the computing device 108 identifies an increase in temperature outside of the region of interest based on video received from the thermographic camera 226).

In a non-limiting example, the video module aiming device 228 may change the view of the thermographic camera 226 to the subject S if the computing device 108 identifies an increase in temperature at the subject S that suggests an incontinence event has occurred. Identifying an increase in temperature in the subject S is desirable because an increase in temperature is often a sign of a patient deterioration event such as an incontinence event, sickness, injury, or infection.

In another non-limiting example, the video module aiming device 228 may change the view of the thermographic camera 226 to a safety hazard 112, such as a spill incident, if the computing device 108 identifies an increase in temperature outside of the subject arrangement area 104. Identifying an increase in temperature relating to a safety hazard 112 is desirable because it allows a caregiver to eliminate the safety hazard 112 before a subject S is harmed in an event relating to the safety hazard 112.

The motion module 230 is configured to capture movements within its view in a video that includes a plurality of images in a chronological sequence. The motion module 230 then transmits the video containing the captured movements to the computing device 108.

In some embodiments, the motion module 230 includes one or more of a motion module video camera 232 and a motion module RGB-D Sensor 234. The motion module video camera 232 and motion module RGB-D sensor operate substantially the same as the video camera 222 and the RGB-D sensor 224 and are configured to capture motion using an additive RGB color model that may include depth imaging.

In some embodiments, the motion module 230 includes a radar sensor 236. The radar sensor 236 is configured to use radar signals to determine various characteristics of objects. For example, range and velocity are used to infer characteristics such as whether an object is accelerating or decelerating or if an object is rotating. Combinations of these motions are used to characterize the activity of the object. For example, the radar sensor 236 is configured to detect movements of an object, such as a subject S, as described herein. In some embodiments, the radar sensor 236 may be used in combination with the video module to estimate the temperature, respiratory rate, or heart rate of a subject S remotely and continuously.

In some embodiments, the radar sensor 236 includes a radar transmission device to transmit radar signals toward an object (e.g., the subject S) and receive reflected radar signals, which can be used to determine the motion of an object or other vital sign measurements such as heart rate and respiration rate. In other examples, LIDAR, ultrasound, sonar, and the like may be used. In certain examples, the radar transmission device includes an antenna.

In some embodiments, the radar sensor 236 operates to transmit the radar signals received from the radar transmission device to the computing device 108 for the computing device 108 to determine a vital sign measurement. In some examples, the radar transmission device is integral with the radar sensor 236.

In alternative embodiments, other types of sensors can be used in addition to or in place of the radar sensor 236. For instance, an infrared array can be used to sense a wide field of view to determine incontinence events in a manner similar to that described herein. Many configurations are possible.

In some embodiments, the motion module 230 includes one more load cells 238. The one or more load cells 238 are configured to detect movement of the subject S by measuring a force exerted by the subject S, converting the force to an electrical signal, and transmitting the electrical signal to the computing device 108 to be analyzed. One or more load cells 238 can be included within the subject arrangement area 104, such as on the bed, to detect movement of the subject S within the subject arrangement area 104. The load cells 238 may be used in conjunction with the video module to improve the confidence of measurements taken by the monitoring device 102.

The motion module 230 includes a motion module aiming device 240 that is configured to aim the motion module 230 at a region of interest by receiving instructions from the computing device 108. Upon receiving the instructions from the computing device 108, the motion module 230 is configured to measure motion within the region of interest relative to the surrounding area. The motion module 230 then provides video of the motion within the region of interest and the surrounding area to the computing device 108 for further analysis.

In some embodiments, the motion module aiming device 240 includes a laser or a light-emitting diode (LED) that emits a visible light in the direction of the radar signals transmitted from the radar transmission device. In this manner, the motion module aiming device 240 assists a medical professional to direct the radar transmission device towards an appropriate region of interest (e.g., the subject arrangement area 104 where the subject S is located or another area where motion is detected). The visible light emitted from the motion module aiming device 240 not only ensures that the radar transmission device is pointed in an appropriate direction, but also provides a visual confirmation to a medical profession that data is being collected.

In some embodiments, the instructions direct the motion module aiming device 240 to a predetermined location that includes the region of interest. The predetermined location can include the subject S, the subject arrangement area 104, the support device 106, or the safety hazard 112.

In some embodiments, the instructions direct the motion module aiming device 240 to a view where the motion module 230 observes motion (i.e., the computing device 108 can instruct the motion module 230 to change its view to a new region of interest if the computing device 108 identifies changes in motion within the region of interest based on video received from the motion module 230). In a non-limiting example, the motion module aiming device 240 may change the view of the motion module 230 to the subject S if the computing device 108 identifies movements from the subject S. Identifying movements from the subject S is desirable because the movements may indicate the subject S is experiencing discomfort. Discomfort may arise from a patient deterioration event, including an incontinence event, a pressure injury, patient sickness, or an infection.

The vital sign module 242 is configured to read the vital signs of the subject S within the subject arrangement area 104. The vital sign module 242 may include an oximeter 244, a heart rate sensor 246, a respiration rate sensor 248, and a temperature sensor 250. The oximeter 244 is configured to measure the subject's blood oxygen saturation. The heart rate sensor is configured to measure the subject's heart rate. The respiration rate sensor 248 is configured to measure the subject's respiration rate. The temperature sensor 250 is configured to measure the temperature of the subject. The vital sign module 242 may be used alone or in conjunction with the video module 220 or the motion module 230 to improve the confidence of measurements taken by the monitoring device 102. Measuring the subject's blood oxygen saturation, heart rate, respiratory rate, and temperature is desirable because it provides early indications of patient deterioration.

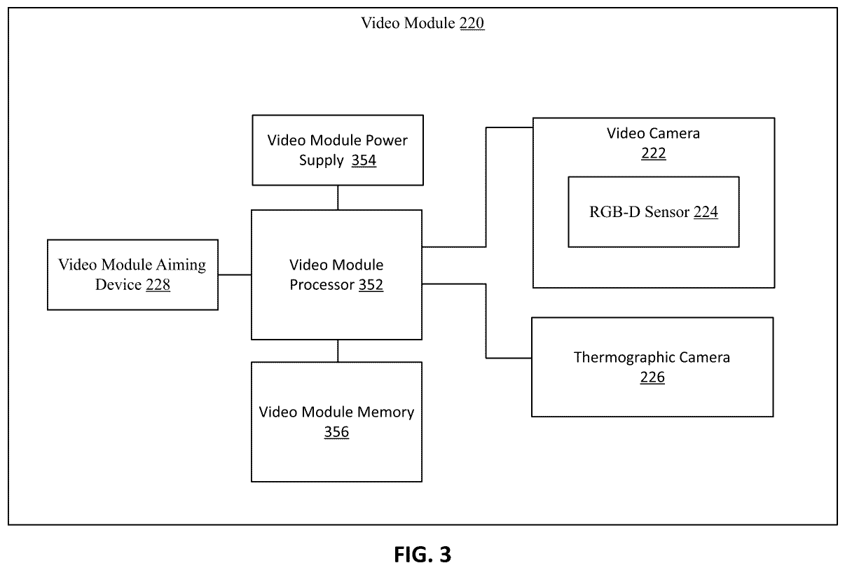

FIG. 3 is a block diagram of the example video module 220 of FIG. 2. The video module 220 includes the video module aiming device 228, the video camera 222, the RGB-D sensor 224, and the thermographic camera 226 of FIG. 2. Furthermore, the video module 220 includes a video module processor 352, a video module memory 356, and a video module power supply 354.

The video module processor 352 is configured to receive and execute instructions from the computing device 108 to operate and control the video camera 222, the thermographic camera 226, and the video module aiming device 228. Furthermore, the video module processor 352 is configured to retrieve and store data within the video module memory 356. In some examples, the video module processor 352 is further configured to perform the functionalities of the video module 220, such as processing and analyzing of the video recorded by the video camera 222 and/or the thermographic camera 226 to detect changes in temperature or behavior within a viewed area.

The video module memory 356 includes one or more memories configured to store data associated with the video recorded by the video camera 222 and/or the thermographic camera 226 and data usable to evaluate the recorded video. The video module memory 356 can be of various types, including volatile and nonvolatile, removable and non-removable, and/or persistent media. In some embodiments, the video module memory 356 is an erasable programmable read only memory (EPROM) or flash memory.

The video module power supply 354 provides power to operate the video module 220 and associated elements. In some examples, the video module power supply 354 includes one or more batteries, which is either for single use or rechargeable. In other examples, the video module power supply 354 includes an external power source, such as mains power or external batteries.

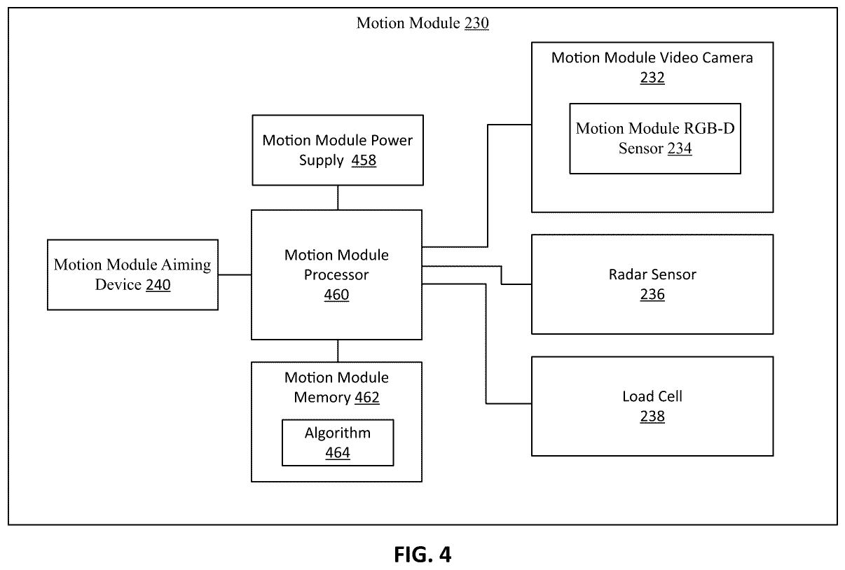

FIG. 4 is a block diagram of the example motion module 230 of FIG. 2. The motion module 230 includes the motion module aiming device 240, the motion module video camera 232, the motion module RGB-D sensor 234, the radar sensor 236, and the load cells 238 of FIG. 2. Furthermore, the motion module 230 includes a motion module processor 460, a motion module memory 462 including an algorithm 464, and a motion module power supply 458.

The motion module processor 460 is configured to receive and execute instructions from the computing device 108 to operate the motion module 230 and its associated components. Furthermore, the motion module processor 460 is configured to retrieve and store data within the motion module memory 462. In some embodiments, this includes executing an algorithm 464. The algorithm 464, when executed by the motion module processor 460, causes the motion module aiming device 240 to electronically steer transmitting antennas to adjust the direction of the radar signal transmission in the azimuth and elevation directions. Additionally, the algorithm 464 causes motion module processor 460 to electronically focus the radar signal transmission by narrowing the dispersion of the radar signals (e.g., from 120 degrees to 40 degrees, 30 degrees, 20 degrees, etc.) to increase the density of the radar signals on a region of interest. In some examples, the video module processor 352 is further configured to perform the functionalities of the video module 220, such as processing and analyzing of the video recorded by the video camera 222 and/or the thermographic camera 226 to detect changes in temperature or behavior within a viewed area.

The motion module memory 462 includes one or more memories configured to store data associated with the video recorded by the motion module 230 and data usable to evaluate the recorded video. The motion module memory 462 can be of various types, including volatile and nonvolatile, removable and non-removable, and/or persistent media. In some embodiments, the video module memory 356 is an erasable programmable read only memory (EPROM) or flash memory. In this example, the motion module memory 462 stores the algorithm 464 that uses the amplitude of the reflected radar signals from the radar sensor 236 to electronically adjust the direction and focus of the radar signal transmission. Alternatively, the algorithm 464 may be stored in a memory of the motion module 230.

The motion module power supply 458 provides power to operate the motion module 230 and associated elements. In some examples, the motion module power supply 458 includes one or more batteries, which is either for single use or rechargeable. In other examples, the motion module power supply 458 includes an external power source, such as mains power or external batteries.

FIG. 5 is a block diagram of an example computing device 108 of FIG. 1. The computing device 108 includes a computing device processor 568 that is configured to retrieve data from the monitoring device 102, including video from the video module 220, video from the motion module 230, or vital sign measurements from the vital sign module 242. Furthermore, the computing device processor 568 is configured to retrieve and store data within the computing device memory 572. In some examples, the computing device processor 568 is further configured to perform the functionalities of the computing device, such as operating artificial intelligence models 570 to process and analyze data retrieved from the monitoring device 102.

The computing device memory 572 includes one or more memories configured to store data associated with the monitoring device, including video module data 574, motion module data 576, and vital sign module data 578. The computing device memory 572 also includes data usable to evaluate the recorded video. The video module data 574 includes video and other data generated from the video module 220. The motion module data 576 includes video and other data generated from the motion module 230. The vital sign module data includes data generated by the vital sign module 242. The computing device memory 572 can be of various types, including volatile and nonvolatile, removable and non-removable, and/or persistent media. In some embodiments, the video module memory 356 is an erasable programmable read only memory (EPROM) or flash memory.

The computing device power supply 566 provides power to operate the computing device 108 and associated elements. In some examples, the computing device power supply 566 includes one or more batteries, which is either for single use or rechargeable. In other examples, the video module power supply 354 includes an external power source, such as mains power or external batteries.

Artificial intelligence can be applied to process data and apply the data to certain applications. For instance, a machine learning algorithm can be trained using data from a large number of subjects. The artificial intelligence includes the artificial intelligence models 570 that are configured to analyze monitoring device data 582 stored in the computing device memory 572 (including the video module data 574, the motion module data 576, and the vital sign module data 578) and apply the data to certain applications.

In an example application, the artificial intelligence models 570 are configured to continuously monitor a region of interest. Methods for defining the region of interest using the computing device 108, and specifically the artificial intelligence models 570, are illustrated and described in further detail with reference to FIG. 2. The computing device memory 572 encodes instructions that, when executed by the computing device processor, cause the computing device to receive data from the monitoring device 102, monitor the region of interest using the artificial intelligence models 570 by analyzing a video feed from the monitoring device 102, detect changes to the region of interest relative to the surrounding area, and issue an alert when the changes exceed a threshold value. The data received from the monitoring device includes one or more of the video module data 574, the motion module data 576, or the vital sign module data 578. The artificial intelligence models 570 are configured to detect changes in temperature and motion within the region of interest relative to the surrounding area.

Furthermore, the artificial intelligence models 570 are configured to detect changes in data received from the monitoring device and issue an alert when the data exceeds a threshold value. In some embodiments, the alert is issued to a notification system 580, which includes a caregiver call system, a patient dashboard, a nurse station, or a mobile application. In some embodiments, the alert includes images of the subject S that depict an increase in temperature or motion that exceeds a threshold value.

In some embodiments, the alert includes a de-identified video clip of the subject S that depicts the increase in temperature or motion that exceeds the threshold value. The de-identified video clip is useful for a caregiver to provide context relating to the reason for issuing the alert. In yet other examples, action in addition to issuing an alert can be taken when an incontinence event is detected, such as triggering the release of desiccant and/or automating the replacement of bed linens.

In some examples, the de-identified video clip can include a thermographic image of the subject arrangement area 104 and the subject S. The monitoring device 102, and specifically the thermographic camera 226, can record at least one image that shows a difference between the temperature of a region of interest, such as the subject S, and a surrounding area, such as a bed. The computing device 108 can then transmit the at least one image to the computing device 108, where the computing device can analyze the at least one image and identify the difference in temperature. After identifying the difference in temperature, the computing device 108 can then send the at least one image that illustrates the difference in temperature to the caregiver to provide context for the alert that is issued. The caregiver can review the at least one image sent from the computing device 108 and understand that an incontinence event has likely occurred, so the caregiver will know to allocate caregiving resources to address the incontinence event.

In other examples, the de-identified video clip can include a plurality of images of the subject arrangement area 104 and the subject S that depicts movement of the subject S. The monitoring device 102, and specifically the motion module video camera 232, can record a plurality of images that show a change in location or orientation of the subject S. Furthermore, the load cells 238 or the radar sensor 236 can record data that indicates a change in location or orientation of the subject S. Data from the motion module video camera 232, the radar sensor 236, or the load cells 238 can be transmitted from the monitoring device 102 to the computing device 108 for further analysis, where the computing device 108 can analyze the data to identify the changes in location or orientation of the subject S. After identifying the changes in location or orientation of the subject S, the computing device 108 can then send the plurality of images that illustrates the changes in location or orientation of the subject S to the caregiver to provide context for the alert that is issued. The caregiver can review the at least one image sent from the computing device 108 and understand that an incontinence event has likely occurred, so the caregiver will know to allocate caregiving resources to address the incontinence event. In certain examples, micromovements (including minute changes in location or orientation of the subject S) can indicate discomfort in the subject S due to increased wetting of the subject's skin or clothes that may contribute to pressure injuries. This discomfort may indicate an incontinence event has occurred.

In some embodiments, the threshold value is determined using vital sign module data 578 or medical records of the subject S. In a non-limiting example, when a subject's average body temperature can be determined from the vital sign module data 578 or from the subject's medical records, increases in temperature could be determined by comparing a region of interest of the subject S to an average body temperature of the subject S.

In another non-limiting example, the artificial intelligence models 570 could determine an average level of activity for a subject S and issue an alert when the artificial intelligence models determine the motion module data indicates the subject S is exhibiting a level of activity above the average level of activity. Such an alert is desirable because increased activity may indicate the subject S is experiencing discomfort. Discomfort may arise from patient deterioration events such as an incontinence event, a sickness, an injury, or an infection.

In another example application, the artificial intelligence models 570 are configured to continuously monitor a region of interest for safety hazard 112. The artificial intelligence models 570 are configured to identify safety hazards by monitoring a region of interest for increases in temperature or movement relative to a threshold value.

In a non-limiting example, the artificial intelligence models 570 can identify a safety hazard 112 by analyzing video module data 574 and motion module data 576 relating to a spill. When a spill occurs, solid or liquid material is dropped to the floor. When this occurs, the video module 220 may detect and record a change in temperature at the location where the spill occurred. Furthermore, the motion module 230 may detect and record a change in motion at the location where the spill occurred.

In an alternative embodiment, the video module data 574 is further analyzed to assess fluid intake and loss. For instance, the artificial intelligence models 570 can be programmed to analyze the video module data 574 to determine fluid intake, such as fluid consumed by the subject S through drinking, intravenous bags, etc. The artificial intelligence models 570 can further be programmed to estimate fluid loss, such as through urine collected in a collection bag, bathroom events, incontinence, etc. The example artificial intelligence models 570 can thereby estimate a total fluid gain or loss for the subject S through such calculations.

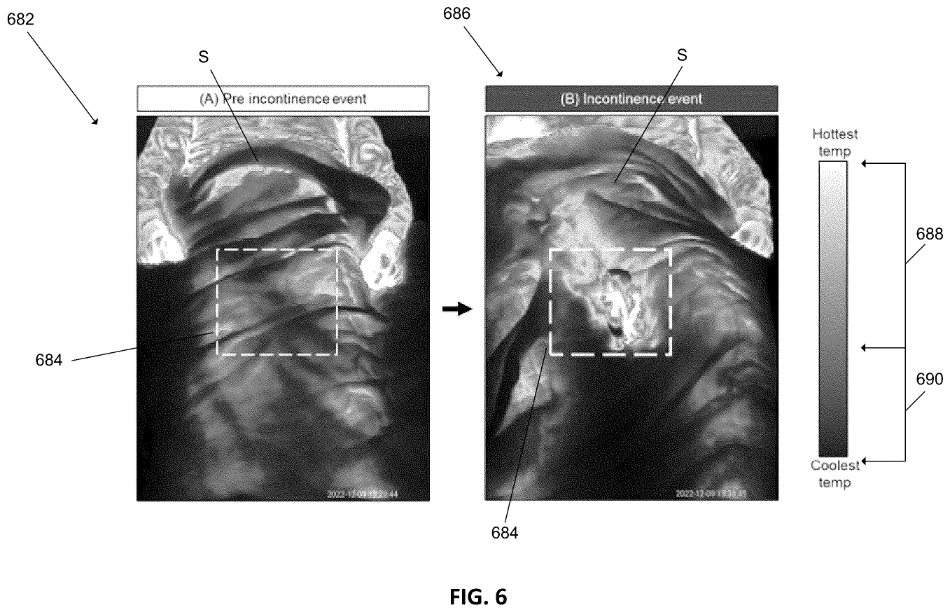

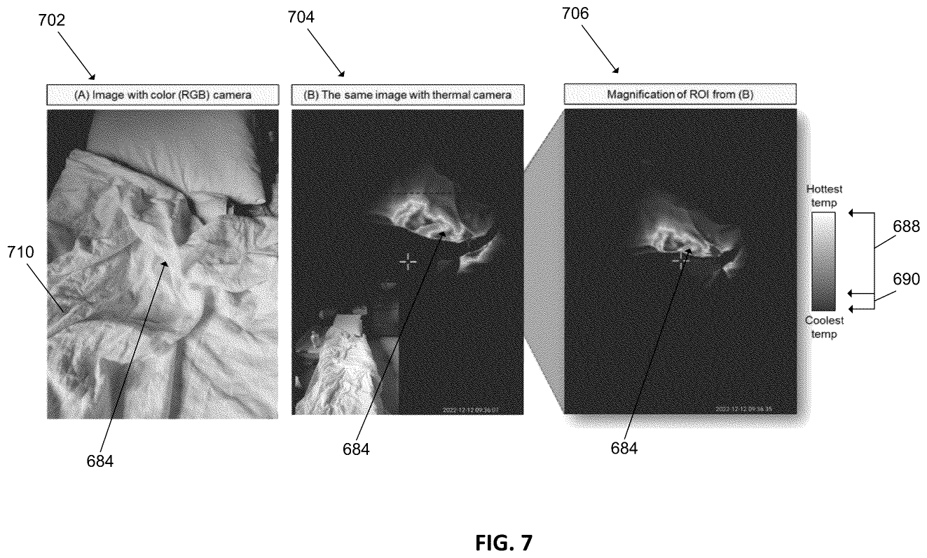

The computing device 108 is configured to receive recorded data from the video module 220 and the motion module 230 to be analyzed by the artificial intelligence models 570. The artificial intelligence models 570 may then compare the increase in temperature or motion at the location where the spill occurred to threshold values to determine a safety hazard 112 is present. After determining a safety hazard 112 is present, the artificial intelligence models 570 can issue an alert to the notification system 580 to notify caregivers of the safety hazard and expedite the process of removing the safety hazard 112. Additional example applications relating to incontinence events are illustrated and described in further detail with respect to FIGS. 6-7.

FIG. 6 illustrates an example incontinence event of a subject S that is detected by the monitoring device 102 of FIG. 2. A pre-incontinence event image 682 is shown as captured by the thermographic camera 226. The pre-incontinence event image 682 includes a region of interest 684 indicated by a dashed shape. The thermographic camera 226 only captures cooler temperatures 690 within the region of interest 684. At this point in time, the subject S has not experienced an incontinence event, and the artificial intelligence models 570 would refrain from issuing an alert to a notification system 580. The region of interest can be defined by the computing device 108 to include certain areas of the subject S. In certain examples, the computing device 108 will define the region of interest to include the subject's mid-section and lower body if the computing device 108 analyzes at least one image received form the thermographic camera 226 that indicates an incontinence event has occurred.

Furthermore, FIG. 6 shows an incontinence event image 686 containing the same region of interest 684 as the pre-incontinence event image 682. The incontinence event image 686 exhibits elevated temperatures 688 within the region of interest 684. This indicates the subject S has likely experienced an incontinence event, and the artificial intelligence models 570 are configured to issue an alert to the notification system 580 to alert caregivers of the incontinence event.

In an alternative embodiment, one or more of the fabrics associated with the subject S can also be used to estimate incontinence events. For instance, the clothing of the subject S (e.g., hospital gown) and/or the sheets or blankets of the support device 106 can be configured to change color upon absorption of fluids. This can thereby be another indicator of an incontinence event.

In a further embodiment, an infrared beacon can be used to communicate the incontinence event. For instance, an infrared beacon can be coupled to the sheets or blankets of the support device 106. Upon detection of fluid by the sheets or blankets, the infrared beacon can be activated (e.g., blink) to communicate the incontinence event to the monitoring device 102. In such scenarios, the infrared beacon could be coupled to the sheets or blankets and could be detected even if covered.

In yet another example, other input can be used to determine an incontinence event. For instance, the monitoring device 102 can be configured to accept a voice input from the subject S and/or a caregiver to indicate that an incontinence event has occurred. For instance, the subject S can orally indicate: “I have soiled my linens.” The monitoring device 102 can use that in conjunction with other inputs (such as video) to capture the incontinence event. In yet other examples, other types of sensors, such as gas sampling detectors (e.g., odor sensors), can also be used to detect the incontinence event. Many configurations are possible.

FIG. 7 illustrates another example incontinence event of the subject S that is detected by the monitoring device of FIG. 2 below a patient covering. In this example, the subject S is not shown in the support apparatus for ease of illustration.

A first RGB image 702 illustrates a patient covering on an example subject arrangement area 104 where an incontinence event has occurred. The first RGB image 702 illustrates the event in an RGB image. In this example, it is difficult for the patient monitoring system 100 to detect an incontinence event within a region of interest 684 if the incontinence event occurs below a patient covering 710 that conceals changes in moisture associated with an incontinence event or other safety hazard.

A first thermographic image 704 illustrates the patient covering on an example subject arrangement area 104 where an incontinence event has occurred. The first thermographic image 704 illustrates the event in a thermographic image. Here, a temperature gradient illustrates elevated temperatures 688 are present in the region of interest 684. Thus, the artificial intelligence models 570 can detect incontinence events below patient coverings by analyzing video module data 574 obtained by the thermographic camera 226.

A second thermographic image 706 illustrates the patient covering on an example subject arrangement area 104 where an incontinence event has occurred. The second thermographic image 706 illustrates the event in a magnified thermographic image. Here, the magnified thermographic image focuses on a portion of the region of interest 684 where elevated temperatures 688 are observed.

For instance, in this example, the ambient environment (including the patient covering 710) is at a first temperature (e.g., 64.8 degrees F.), while the incontinence event is at a higher second temperature (e.g., 89.4 degrees F.). The artificial intelligence models 570 can be programmed to detect this change in temperature.

The artificial intelligence models 570 can magnify incontinence events to further aid caregivers in identifying incontinence events and caring for subjects S. For instance, as noted above, a de-identified image can be provided to the caregiver (e.g., at a call station) in conjunction with an alert for the incontinence event so that the caregiver can determine a potential location for the event.

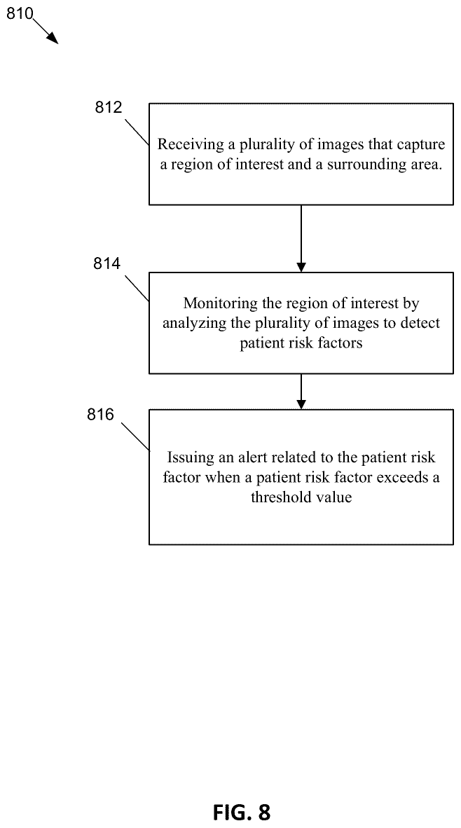

FIG. 8 illustrates a method for detecting and mitigating patient deterioration 810.

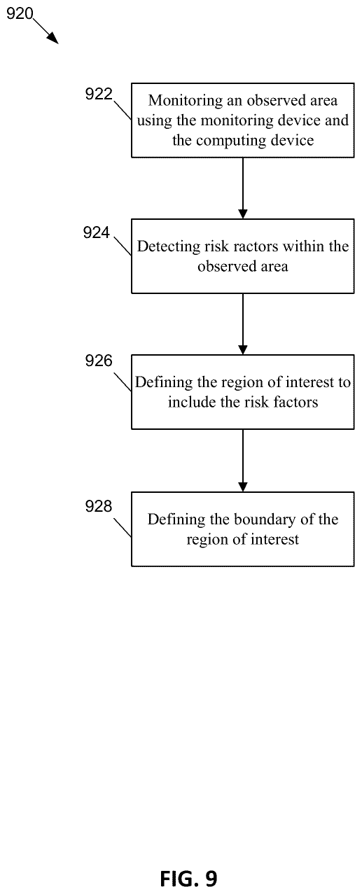

The method 810 includes an initial step 812 of using the computing device 108 to receive a video feed from the monitoring device 102 that includes a region of interest and a surrounding area. The process of defining the region of interest and the surrounding area is illustrated and described in further detail with reference to FIGS. 2 and 9.

The method 810 includes a second step 814 of monitoring the region of interest, using the computing device 108, by analyzing the plurality of images received from the monitoring device 102 to detect patient risk factors. The computing device 108 analyzes the plurality of images using artificial intelligence models 570 to detect patient deterioration events within the region of interest. The artificial intelligence models 570 can identify patient deterioration events by identifying locations where an increase in temperature or motion exceeds a threshold a value.

The method 810 includes a third step 816 of issuing an alert related to the patient deterioration event when a patient risk factor exceeds a threshold value. Examples of patient risk factors include an increase in temperature, an increase in motion, a decrease in a patient's blood oxygen saturation, or a significant increase or decrease in a patient's heart rate or respiration rate. Methods of issuing an alert related to the patient deterioration event and determining an appropriate threshold value are illustrated and described in further detail with reference to FIG. 5.

FIG. 9 illustrates a method of associating a region of interest with one or more patient risk factors 920. The method 920 includes a first step 922 of monitoring an observed area using the monitoring device 102 and the computing device 108. The monitoring device is configured to obtain data within the observed area, such as video, and transmit the data to the computing device 108 for further analysis. The computing device 108 then analyzes the data received from the monitoring device 102 as it is received in real time to continuously monitor the observed area. The observed area includes the areas where data is being collected, such as the view of the video module 220 or motion module or the subject S that is being monitored by the vital sign module 242.

The second step 924 includes detecting risk factors within the observed area. As illustrated and described in FIG. 8, examples of patient risk factors include an increase in temperature, an increase in motion, a decrease in a patient's blood oxygen saturation, or a significant increase or decrease in a patient's heart rate or respiration rate. The monitoring device 102 is used to record video and other data to detect patient risk factors within the observed area.

The third step 926 includes defining the region of interest to include the risk factors that are detected in the second step 924. In a non-limiting example, if an increase in temperature is observed due to an incontinence event, then the region of interest would be defined to include the increase in temperature, and specifically the subject S. The fourth step 928 includes defining the boundaries of the region of interest, which thereby identifies the area surrounding the region of interest. In this example, this might include objects surrounding the patient, such as a bed.

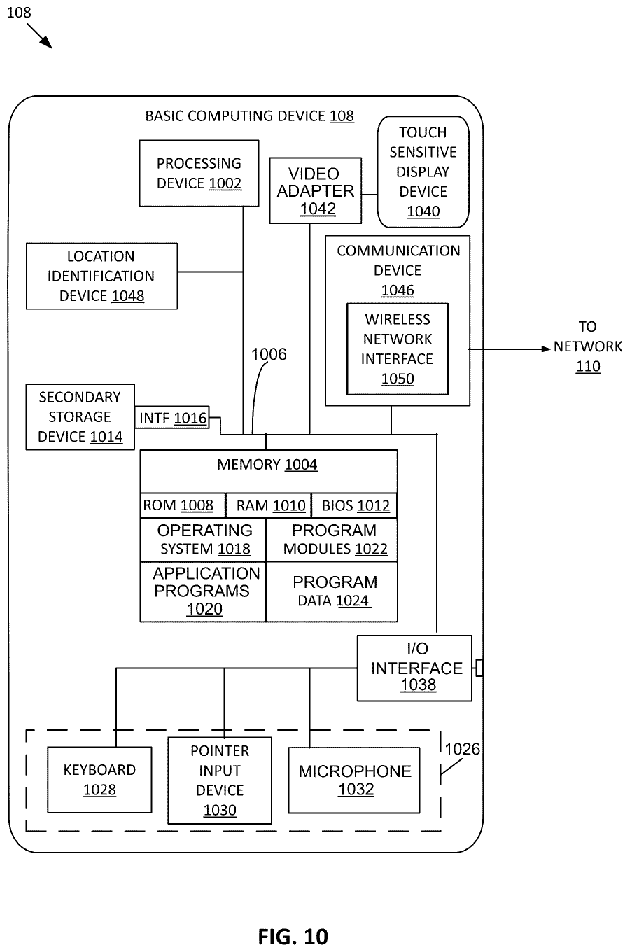

FIG. 10 illustrates an exemplary architecture of the computing device 108. The monitoring device 102 and other devices described herein can be similarly configured.

The computing device 108 is used to execute the operating system, application programs, and software modules (including the software engines) described herein. The monitoring device 102 and the computing device 108 can include all or some of the elements described with reference to FIG. 10, with or without additional elements.

The computing device 108 includes, in some examples, at least one processing device 1002, such as a central processing unit (CPU). A variety of processing devices are available from a variety of manufacturers, for example, Intel or Advanced Micro Devices. In this example, the computing device 108 also includes a system memory 1004, and a system bus 1006 that couples various system components including the system memory 1004 to the processing device 1002. The system bus 1006 is one of any number of types of bus structures including a memory bus, or memory controller; a peripheral bus; and a local bus using any of a variety of bus architectures.

The system memory 1004 may include read only memory 1008 and random access memory 1010. A basic input/output system 1012 containing the basic routines that act to transfer information within the computing device 108, such as during start up, is typically stored in the read only memory 1008.

The computing device 108 also includes a secondary storage device 1014 in some embodiments, such as a hard disk drive, for storing digital data. The secondary storage device 1014 is connected to the system bus 1006 by a secondary storage interface 1016. The secondary storage devices and their associated computer readable media provide nonvolatile storage of computer readable instructions (including application programs and program modules), data structures, and other data for the computing device 108.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 1014 or memory 1004, including an operating system 1018, one or more application programs 1020, other program modules 1022, and program data 1024.

In some embodiments, the computing device 108 includes input devices to enable a user to provide inputs to the computing device 108. Examples of input devices 1026 include a keyboard 1028, a pointer input device 1030, a microphone 1032, and a touch sensitive display 1040. Other embodiments include other input devices. The input devices are often connected to the processing device 1002 through an input/output interface 1038 that is coupled to the system bus 1006. These input devices 1026 can be connected by any number of input/output interfaces, such as a parallel port, serial port, game port, or a universal serial bus. Wireless communication between input devices and the input/output interface 1038 is possible as well, and includes infrared, BLUETOOTH® wireless technology, 802.11a/b/g/n, cellular, or other radio frequency communication systems in some possible embodiments.

In one example embodiment, a touch sensitive display 1040 is connected to the system bus 1006 via an interface, such as a video adapter 1042. The touch sensitive display 1040 includes touch sensors for receiving input from a user when the user touches the display. Such sensors can be capacitive sensors, pressure sensors, or other touch sensors. The sensors detect contact with the display, and also the location and movement of the contact over time. For example, a user can move a finger or stylus across the screen to provide written inputs. The written inputs are evaluated and, in some embodiments, converted into text inputs.

In addition to the touch sensitive display 1040, the computing device 108 can include various other peripheral devices (not shown), such as speakers or a printer.

The computing device 108 further includes a communication device 1046 configured to establish communication across a network. In some embodiments, when used in a local area networking environment or a wide area networking environment (such as the Internet), the computing device 108 is typically connected to the network through a network interface, such as a wireless network interface 1050. Other possible embodiments use other wired and/or wireless communication devices. For example, some embodiments of the computing device 108 include an Ethernet network interface, or a modem for communicating across the network.

In yet other embodiments, the communication device 1046 is capable of short-range wireless communication. Short-range wireless communication is one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

The computing device 108 typically includes at least some form of computer-readable media. Computer readable media includes any available media that can be accessed by the computing device 108. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 108. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device 108 is an example of programmable electronics, which may include one or more computing devices, and when multiple computing devices are included, such computing devices can be coupled together with a suitable data communication network so as to collectively perform the various functions, methods, or operations disclosed herein.

The computing device 108 can include a location identification device 1048. The location identification device 1048 is configured to identify the location or geolocation of the computing device 108. The location identification device 1048 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

Embodiments of the present invention may be utilized in various distributed computing environments where tasks are performed by remote processing devices that are linked through a communications network in a distributed computing environment.

The block diagrams depicted herein are just examples. There may be many variations to these diagrams described therein without departing from the spirit of the disclosure. For instance, components may be added, deleted or modified.

The systems and method described herein result in a significant technical advantage. For example, the monitoring device 102 more efficiently captures video module data, motion module data, and vital sign data. Additionally, the computing device 108 monitors patients' temperature, movements, and vital sign measurements more accurately and in less time. Furthermore, the captured information can be more efficiently communicated to caregivers in an alert system that prompts early detection of patient deterioration.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless of whether shown and described in combination or separately, the various features (both structural and methodological) are intended to be selectively included or omitted to produce an embodiment with a particular set of features.

Having been provided with the description and illustration of the present application, one skilled in the art may envision variations, modifications, and alternate embodiments falling within the spirit of the broader aspects of the claimed inventions and the general inventive concepts embodied in this application that do not depart from the broader scope.

What is claimed is:

1. A patient monitoring system, comprising:
a computing device including a processor and a memory, the memory encoding instructions that, when executed by the processor, cause the system to:
receive, from a thermographic camera, at least one image corresponding to a temperature of a region of interest and a surrounding area that surrounds the region of interest;
analyze the at least one image using an artificial intelligence model;
detect a difference in temperature of the region of interest relative to the surrounding area; and
issue an alert related to an incontinence event when the difference in temperature exceeds a threshold value.

2. The patient monitoring system of claim 1, wherein the instructions, when executed, further cause the system to generate a video, using the artificial intelligence model and the at least one image, which displays the difference in temperature.

3. The patient monitoring system of claim 1, wherein the region of interest comprises a patient.

4. The patient monitoring system of claim 3, wherein the region of interest is the patient's lower body, forehead, or arms.

5. The patient monitoring system of claim 4, wherein the region of interest comprises a patient covering.

6. The patient monitoring system of claim 5, wherein the patient covering comprises clothes, a patient bedsheet, or a patient blanket.

7. The patient monitoring system of claim 3, wherein the instructions, when executed, further cause the system to automatically perform a patient deterioration mitigation action at a patient support apparatus.

8. The patient monitoring system of claim 7, wherein the patient deterioration mitigation action comprises turning the patient at pre-determined time intervals after issuing the alert.

9. The patient monitoring system of claim 7, wherein the patient deterioration mitigation action comprises detecting moisture within the region of interest and drying the region of interest when moisture is detected.

10. The patient monitoring system of claim 1, further comprising a caregiver call system configured to receive the alert.

11. The patient monitoring system of claim 10, wherein the caregiver call system comprises a patient user interface on the patient monitoring system, a nurse station, or a mobile device user interface.

12. The patient monitoring system of claim 1, comprising further instructions that, when executed by the processor, cause the system to:
monitor an observed area including the region of interest and the surrounding area;
measure a temperature of objects within the observed area;
detect an increase in the temperature of objects within the observed area; and
define the region of interest to include the increase in the temperature.

13. A method for detecting patient deterioration, comprising:
receiving, from a thermographic camera, at least one image corresponding to a temperature of a region of interest and a surrounding area that surrounds the region of interest;
using an artificial intelligence model, analyzing the at least one image;
detecting a difference in temperature of the region of interest relative to the surrounding area; and
issuing an alert related to an incontinence event when the difference in temperature exceeds a threshold value.

14. The method of claim 13, further comprising:
receiving, from a motion module, a plurality of images corresponding to a motion within the region of interest and the surrounding area that surrounds the region of interest;
using the artificial intelligence model, analyzing the plurality of images;
detecting the motion within the region of interest relative to the surrounding area; and
issuing the alert related to the incontinence event when the motion exceeds the threshold value.

15. The method of claim 14, wherein the motion module comprises a video camera, a radar sensor, or a red, green, blue plus depth camera (RGB-D).

16. The method of claim 13, further comprising:
receiving, from a vital sign module, real-time vital sign data corresponding to a patient;
using the artificial intelligence model, analyzing the real-time vital sign data;
detecting a change in the patient's vital signs relative to baseline vital sign data corresponding to the patient; and
issuing the alert when a difference between the real-time vital sign data and the baseline vital sign data exceeds the threshold value.

17. The method of claim 13, further comprising automatically performing a patient deterioration mitigation action.

18. The method of claim 17, further comprising turning a patient, using a patient support apparatus, at a pre-determined time intervals to mitigate pressure injuries.

19. The method of claim 17, further comprising detecting moisture within the region of interest and providing heat to the region of interest when moisture is detected.

20. The method of claim 13, further comprising generating a video, using the artificial intelligence model and the at least one image, which displays the difference in temperature.
